(19) United States
(12) Patent Application Publication
(10) Pub. No.: US 2025/0027895 A1
(43) Pub. Date: Jan. 23, 2025

(54) Gas Sensor Arrays and Methods of Fabrication Thereof

(71) Applicant: National University of Singapore, Singapore (SG)

(72) Inventors: Gokula Krishnan RAMACHANDRAN, Singapore (SG); Chen Hua YEOW, Singapore (SG)

(21) Appl. No.: 18/715,689

(22) PCT Filed: Dec. 2, 2022
(86) PCT No.: PCT/SG2022/050878

§ 371 Date: May 31, 2024

(30) Foreign Application Priority Data
Dec. 2, 2021 (SG) 10202113401P

(51) Int. Cl. G01N 27/12; G01N 33/00

(52) U.S. Cl. CPC G01N 27/126; G01N 33/0027

(57) ABSTRACT

The present disclosure concerns gas sensor and gas sensor arrays and their methods of fabrication. The gas sensor array comprises a chemiresistor array, each chemiresistor comprising a sensing material; a space adjacent to the sensing material in each chemiresistor, the space for providing the VOC to the sensing material; and electronic means in electrical communication with the chemiresistors in order to detect a change in an electrical property of the sensing material upon exposure to the VOC. Each of the sensing material independently comprises a plant extract, each of the plant extract comprises a plant sensory compound at a concentration of about 1 ng/cm³ to about 100 μg/cm³; and each of the plant extract is electrically resistive, the electrical resistance is configured to change in response to the VOC in the space. The present disclosure also concerns methods of extracting biomolecules from a plant sample.

### CROSS-REFERENCE TO RELATED APPLICATIONS

This patent application is a US National Phase Entry under 371 National Stage Entry of 35 U.S.C. §371 of co-pending International Patent Application No. PCT/SG2022/050878 filed Dec. 2, 2022, titled “Gas Sensor Arrays and Methods of Fabrication Thereof”, which claims priority to Singaporean Patent Application No. 10202113401P filed Dec. 2, 2021, the contents of which are incorporated by reference herein, for all purposes, in its entirety.

### TECHNICAL FIELD

The present invention relates, in general terms, to gas sensor arrays and their methods of fabrication. The present invention also relates to methods of extracting biomolecules from a plant sample.

### BACKGROUND

In all industries, odour assessment is usually performed by human sensory analysis, by chemosensors, or by gas chromatography. The latter technique gives information about volatile organic compounds but the correlation between analytical results and mean odour perception is not direct due to potential interactions between several odorous components.

Olfaction in animals depend on their nose. The nose is a very delicate chemical sensor that can detect and discriminate between various gases. This is based on the nose having an array of different chemical receptors that act as sensors and the ability of the animal to recognise patterns in the brain, which are stimulated by the number and/or type of receptors that are activated or blocked. One receptor can respond to many gases, and one gas can stimulate many receptors in the nose.

Many efforts have been made to replicate this olfaction using chemical sensors. For example, volatile chemical sensor arrays are fabricated as electronic noses (e-noses) that can emulate the olfactory system of animals. In electronic noses, the odours can be characterised using the pattern obtained from the responses of all the individual chemical sensors in the electronic nose. The commonly used chemical sensors in e-nose are known as chemiresistors.

Conducting polymer composites, intrinsically conducting polymers and metal oxides are commonly used sensing materials in the chemiresistive sensors. In the case of metal oxide sensors, in particular, a heater is used as the high temperature is the prerequisite for the proper functioning of these sensors, thus making these sensors less energy efficient. Conducting polymer composite based sensors are another class of chemiresistive sensors that uses conducting particles interspersed in insulating polymer matrices. Most of the constituents of the conducting polymer composite based sensors are either expensive or hazardous. For instance, the most common conducting particle used is polypyrrole, which is prepared by the polymerization of pyrrole using phosphomolybdic acid in a solution of insulating polymer. The resistance changes of polypyrrole sensors in response to the odour molecules is difficult to predict as the chemical interaction of the odour molecules with the conducting polymer can also change in the intrinsic conductivity of the polypyrrole molecules. Another common type is the carbon black based composite sensor, which is hazardous as carbon black is a well-known carcinogen.

Nanomaterials such as functionalized gold nanoparticles, graphene and carbon nanotubes (CNTs) were also used as chemiresistors. The drawbacks of these materials are that they are very expensive and requires hazardous chemicals and conditions for their preparation. Moreover, pristine graphene and CNTs were not sensitive enough for the chemiresistive applications. Hence additional step of creating defects in graphene and CNTs were required to enhance sensitivity.

All existing chemiresistors are expensive or use hazardous chemicals and physical conditions or energy inefficient. It would be desirable to overcome or ameliorate at least one of the above-described problems.

### SUMMARY

The present disclosure is predicated on the understanding that chemiresistors made from plant extracts can be renewable, low-cost and environmentally friendly. The chemiresistors are also to discriminate between different food (such as different flavors of smoothies), their spoilage/fermentation progression (such as tracing the conversion of milk to yogurt), and for disease diagnostics (such as gastric cancer). The sensor can spontaneously recover without any external stimulus, thus making the sensor reusable.

The present disclosure provides a volatile organic compound (VOC) sensor, comprising:
a) a chemiresistor comprising a sensing material;
b) a space adjacent to the sensing material in the chemiresistor, the space for providing the VOC to the sensing material; and
c) electronic means in electrical communication with the chemiresistors in order to detect a change in an electrical property of the sensing material upon exposure to the VOC;
   wherein the sensing material independently comprises a plant extract;
   wherein the plant extract comprises a plant sensory compound at a concentration of about 1 ng/cm³ to about 100 μg/cm³; and
   wherein each of the plant extract is electrically resistive, the electrical resistance is configured to change in response to the VOC in the space.

The present disclosure provides a volatile organic compound (VOC) sensor array, comprising:
a) a chemiresistor array, each chemiresistor comprising a sensing material;
b) a space adjacent to the sensing material in each chemiresistor, the space for providing the VOC to the sensing material; and
c) electronic means in electrical communication with the chemiresistors in order to detect a change in an electrical property of the sensing material upon exposure to the VOC;
   wherein each of the sensing material independently comprises a plant extract;
   wherein each of the plant extract comprises a plant sensory compound at a concentration of about 1 ng/cm³ to about 100 μg/cm³; and
   wherein each of the plant extract is electrically resistive, the electrical resistance is configured to change in response to the VOC in the space.

In some embodiments, the plant sensory compound has a concentration of about 1 ng/cm³ to about 1 μg/cm³.

In some embodiments, each of the plant extract is independently characterised by a dielectric constant of about 1 to about 200.

In some embodiments, the plant extract comprises a protein at a concentration of less than about 30 mg/g.

In some embodiments, the plant extract comprises a carbohydrate at a concentration of less than about 700 mg/g.

In some embodiments, the plant extract is derived from a flowering plant selected from Caesalpinia pulcherrima, Ixora coccinea, or a combination thereof.

In some embodiments, the plant extract is derived from plant part selected from sepal, petal, leaf, root, or a combination thereof.

In some embodiments, the plant extract comprises tannins.

In some embodiments, the plant extract comprises tannin at a concentration of about 0.1 μg/cm³ to about 1000 g/cm³.

In some embodiments, the sensing material is characterised by a thickness of about 0.1 μm to about 100 μm.

In some embodiments, the VOC sensor array further comprises a conductive material sandwiched between and in electrical communication with the chemisresistor array and the electronic means.

In some embodiments, the conductive material is wire glue, carbon grease, carbon nanomaterial (such as graphene, carbon nanotube), or a combination thereof.

In some embodiments, the chemiresistor array comprises at least 2 chemiresistors.

In some embodiments, the space is an enclosed space.

In some embodiments, the electronic means comprises a voltage divider.

In some embodiments, the electronic means comprises a micro controller.

In some embodiments, the change in electrical property is a change in resistance.

In some embodiments, the change in electrical property is a change in capacitance.

In some embodiments, the change in electrical property is obtainable after about 1 min to about 120 min.

In some embodiments, the VOC sensor array further comprises a sample holder for containing a sample, the sample holder in fluid communication with the space.

In some embodiments, the sample holder comprises a heating means and/or an atomiser.

In some embodiments, the VOC sensor array further comprises a flexible substrate or a rigid substrate.

In some embodiments, the flexible substrate is a polyethylene terephthalate (PET) sheet and the rigid substrate is printed circuit board (PCB).

In some embodiments, the interaction between the sensing material and VOC is reversible.

In some embodiments, the VOC sensor array is characterised by a sensitivity of about 80% to about 100%.

The present disclosure also provides a method of fabricating a volatile organic compound (VOC) sensor array as disclosed herein, comprising:
a) casting the plant extract as a layer on the electronic means; and
b) drying the plant extract in order to form the sensing material in electrical communication with the electronic means.

In some embodiments, the extraction step comprises:
a) crushing a plant sample and incubating the plant sample in a polar solvent;
b) separating the plant sample of step a) into a supernatant and a residue; and
c) extracting the supernatant in order to form the plant extract.

In some embodiments, the polar solvent is ethanol.

In some embodiments, the extraction step further comprises:
d) incubating the plant extract in an aprotic solvent;
e) separating the plant extract of step d) into a supernatant and a residue; and
f) extracting the supernatant in order to form a purified plant extract.

In some embodiments, the aprotic solvent is acetonitrile.

In some embodiments, the method further comprises providing a space adjacent to the sensing material in each chemiresistor.

In some embodiments, the method further comprises electrically connecting the conductive material to electronic means.

In some embodiments, the method further comprises a step of normalising the plant extract or purified plant extract against a standard and/or a control.

In some embodiments, the control is a total protein concentration.

In some embodiments, the control is a weight ratio relative to the weight of the plant sample.

### BRIEF DESCRIPTION OF THE DRAWINGS

Embodiments of the present invention will now be described, by way of non-limiting example, with reference to the drawings in which:

FIG. 1 is a schematic representation of the chemiresistor fabrication;

FIG. 2 is a schematic representation of the gas sensor array;

FIG. 3 shows examples of gas sensor arrays;

FIG. 4 is a schematic representation of the working of chemiresistor;

FIG. 5 is a schematic representation of detecting different analytes using a single chemiresistor;

FIG. 6 is a schematic representation of analyte fingerprinting using VOC sensor array;

FIG. 7 shows chemiresistive responses of the chemiresistors and control when exposed to different flavors of smoothies;

FIG. 8 shows radar plots of the distinct pattern of the VOC sensor array responses when exposed to different flavors of smoothies; and

FIG. 9 shows chemiresistive responses of the chemiresistors and control to the conversion of milk to yogurt.

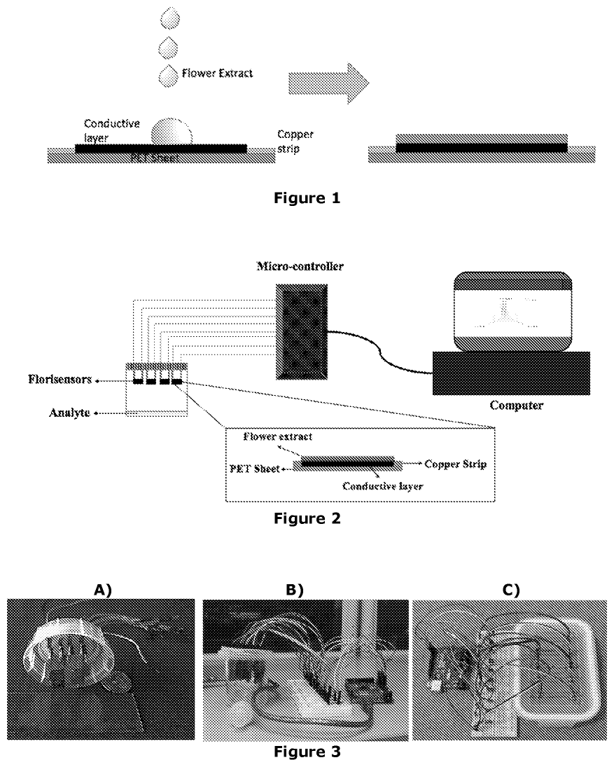

### DETAILED DESCRIPTION

Chemiresistive sensors (or chemiresistors) change their electrical resistances in response to a change in their chemical environment. Chemiresistive sensors consist of a sensing material coated across the metal electrodes. When chemical vapours are exposed to these sensors, the vapours can percolate into the sensing materials and results in a physical change of the sensing material such as swelling. The sensing material and the vapour can interact by covalent bonding, hydrogen bonding, or molecular recognition. This swelling alters the resistivity of the sensing material, thus increasing the resistance. The extent of percolation is depended on the partition coefficient of the gaseous vapour to the corresponding sensing material.

The present disclosure is based on the finding that plant based extracts can be used as a component in the sensing material for chemiresistors. This is predicated on the understanding that herbivore-induced volatiles released by damaged leaves act as volatile cues for the undamaged leaves for enhancing their defences against herbivore damage. Plants, when exposed to a green leaf volatile such as cis-3-hexenyl acetate, result in the priming of the defence genes that mediate oxylipin signaling and direct defences and defence metabolites like jasmonic acid and linolenic acid. Cuscuta pentagona, parasitic plant of Lycopersicon esculentum (Tomato) used the volatile cues for identifying the location of the host. Tobacco plant suffered lesser herbivore attack when grown near the clipped sagebrush than the tobacco plants grown near unclipped sagebrush. An interesting study shows that lima beans produced more extrafloral nectar for attracting predators in response to the herbivore attack. These clearly suggested that plants can perform olfaction, which means plants can detect and discriminate volatile compounds and respond to these volatile cues. Hence, the inventors believe that plant extracts can be utilized as chemiresistive sensing material in volatile sensors. As plant extracts are low-cost, non-hazardous, environmentally friendly and renewable, plant extracts have high potential in substituting energy inefficient, expensive and hazardous chemiresistive sensing materials. When chemiresistors are formed with the sensing material of the present invention, the percolation phenomenon is reversible, thus making these sensors reusable.

The present disclosure is applicable in quality testing in food and essence industries like manuka honey testing, oil and fats quality testing, high value products such as wine and beverage. The present disclosure is applicable for use quality control of fragrances such as perfume and deodorant. The present disclosure is applicable in monitoring gas and volatile compound in places like mines, caves, and in chemical warfare. The present disclosure is applicable as a breath testing device to identify volatile biomarkers of diseases and disorders, as well as in treatment response and disease progression monitoring.

The present disclosure provides a volatile organic compound (VOC) sensor, comprising:
a) a chemiresistor comprising a sensing material;
b) a space adjacent to the sensing material in the chemiresistor, the space for providing the VOC to the sensing material; and
c) electronic means in electrical communication with the chemiresistor in order to detect a change in an electrical property of the sensing material;
   wherein the sensing material independently comprises a plant extract.

The present disclosure provides a volatile organic compound (VOC) sensor array, comprising:
a) a chemiresistor array, each chemiresistor comprising a sensing material;
b) a space adjacent to the sensing material in each chemiresistor, the space for providing the VOC to the sensing material; and
c) electronic means in electrical communication with the chemiresistor in order to detect a change in an electrical property of the sensing material;
   wherein each of the sensing material independently comprises a plant extract.

In some embodiments, the volatile organic compound (VOC) sensor array, comprises:
a) a chemiresistor array, each chemiresistor comprising a sensing material;
b) a space adjacent to the sensing material in each chemiresistor, the space for providing the VOC to the sensing material; and
c) electronic means in electrical communication with the chemiresistor in order to detect a change in an electrical property of the sensing material;
   wherein each of the sensing material independently comprises a plant extract; and
   wherein each of the plant extract comprises a plant sensory compound at a concentration of about 1 ng/cm³ to about 100 μg/cm³.

In some embodiments, the volatile organic compound (VOC) sensor array, comprises:
a) a chemiresistor array, each chemiresistor comprising a sensing material;
b) a space adjacent to the sensing material in each chemiresistor, the space for providing the VOC to the sensing material; and
c) electronic means in electrical communication with the chemiresistor in order to detect a change in an electrical property of the sensing material upon exposure to the VOC;
wherein each of the sensing material independently comprises a plant extract;
   wherein each of the plant extract comprises a plant sensory compound at a concentration of about 1 ng/cm³ to about 100 μg/cm³; and
   wherein each of the plant extract is electrically resistive, the electrical resistance is configured to change in response to the VOC in the space.

In some embodiments, the volatile organic compound (VOC) sensor array, comprises:
a) a chemiresistor array, each chemiresistor comprising a sensing material;
b) a space adjacent to the sensing material in each chemiresistor, the space for providing the VOC to the sensing material; and
c) electronic means in electrical communication with the chemiresistor in order to detect a change in an electrical property of the sensing material;
   wherein each of the sensing material independently comprises a plant extract; and
   wherein each of the plant extract is independently characterised by a dielectric constant of about 1 to about 200.

In some embodiments, the volatile organic compound (VOC) sensor array, comprises:
a) a chemiresistor array, each chemiresistor comprising a sensing material;
b) a space adjacent to the sensing material in each chemiresistor, the space for providing the VOC to the sensing material; and
c) electronic means in electrical communication with the chemiresistor in order to detect a change in an electrical property of the sensing material;
   wherein each of the sensing material independently comprises a plant extract;
   wherein each of the plant extract comprises a plant sensory compound at a concentration of about 1 ng/cm³ to about 100 μg/cm³; and
   wherein each of the plant extract is independently characterised by a dielectric constant of about 1 to about 200.

The ability of the plants to sense its surrounding is found in all plants. In this regard, plant extract from all plants can be used. As plant extracts from all plants can be used, the dimensionality of the VOC sensor array can be further improved by using different combination of plant extracts, or even extracts from different parts of plants. This allows a huge array of gas sensors each have varied responses (resistance as well as capacitance) for different analyte samples, thus enabling generation of a response library specific to different analytes.

As used herein, “plant extract” is a substance made by extracting a part of a plant. The extract can be obtained by extracting from the feedstock, such as blossoms, fruit, and roots, or from intact plants through multiple techniques and methods. For example, expression (juicing, pressing) involves physical extraction material from feedstock, used when the oil is plentiful and easily obtained from materials such as citrus peels, olives, and grapes; absorption (steeping, decoction) is done by soaking material in a solvent, as used for vanilla beans or tea leaves; maceration, as used to soften and degrade material without heat, normally using oils, such as for peppermint extract and wine making; distillation or separation, to create a higher concentration of the extract by heating material to a specific boiling point, then collecting this and condensing the extract, leaving the unwanted material behind, as used for lavender extract.

In some embodiments, the plant extract is derived from a plant species. The plant extract may be derived from a part of the plant or from a combination of different parts of the plant. In some embodiments, the plant extract is derived from various plant species for example and not limited to Rosaceae sp., Canna indica, Bougainvilla glabara, Bougainville spectabillis, Tabernaemontana divaricate, Jasminum officinale, Tagetes erecta, Tagetes patula, Chrysanthemum indium, Caesalpinia pulcherrima, Ixora coccinea Senna Auriculata, Hibiscus rosa-sinensis.

In some embodiments, the plant extract is derived from a flowering plant selected from Caesalpinia pulcherrima, Ixora coccinea, or a combination thereof.

In some embodiments, the plant extract is derived from plant part selected from sepal, petal, leaf, root, or a combination thereof. In other embodiments, the plant extract is derived from plant part selected from sepal, petal, leaf, or a combination thereof. In other embodiments, the plant extract is derived from plant part selected from sepal, petal, or a combination thereof.

It was found that different plant parts have different amounts of plant sensory compound, and possible at different combinations and/or mixtures. This can in particular be advantageous as it can give a unique response or sensing ability to any single VOC. Alternatively, the plant extract may be further purified in order to isolate a single or a mixture of plant sensory compound(s).

“Plant sensory compound” as used herein refers to compounds innate to plants and which allows the plant to respond to volatile cues from the environment. For example, hormones in plants can be used to coordinate plant development and morphology. Electrophysiological signals in plants can be generated through the change in concentration of ions and/or charge carrying molecules to influence processes such as actin-based cytoplasmic streaming, plant organ movements, wound responses, respiration, photosynthesis and flowering, or in response to insects.

In some embodiments, the plant extract comprises a mixture of plant sensory compounds. In some embodiments, the plant extract comprises a plant sensory compound at a concentration of about 0.1 ng/cm³ to about 1000 μg/cm³. In other embodiments, the concentration is about 0.1 ng/cm³ to about 900 μg/cm³, about 0.1 ng/cm³ to about 800 μg/cm³, about 0.1 ng/cm³ to about 700 μg/cm³, about 0.1 ng/cm³ to about 600 μg/cm³, about 0.1 ng/cm³ to about 500 μg/cm³, about 0.1 ng/cm³ to about 400 μg/cm³, about 0.1 ng/cm³ to about 300 μg/cm³, about 0.1 ng/cm³ to about 200 μg/cm³, about 0.1 ng/cm³ to about 100 μg/cm³, about 0.1 ng/cm³ to about 90 μg/cm³, about 0.1 ng/cm³ to about 80 μg/cm³, about 0.1 ng/cm³ to about 70 μg/cm³, about 0.1 ng/cm³ to about 60 μg/cm³, about 0.1 ng/cm³ to about 50 μg/cm³, about 0.1 ng/cm³ to about 40 μg/cm³, about 0.1 ng/cm³ to about 30 μg/cm³, about 0.1 ng/cm³ to about 20 μg/cm³, about 0.1 ng/cm³ to about 10 μg/cm³, or about 0.1 ng/cm³ to about 1 μg/cm³. In other embodiments, the concentration is about 1 ng/cm³ to about 1000 ng/cm³, about 1 ng/cm³ to about 900 ng/cm³, about 1 ng/cm³ to about 800 ng/cm³, about 1 ng/cm³ to about 700 ng/cm³, about 1 ng/cm³ to about 600 ng/cm³, about 1 ng/cm³ to about 500 ng/cm³, about 1 ng/cm³ to about 400 ng/cm³, about 1 ng/cm³ to about 300 ng/cm³, about 1 ng/cm³ to about 200 ng/cm³, or about 1 ng/cm³ to about 100 ng/cm³. In other embodiments, the concentration is about 10 ng/cm³ to about 1000 ng/cm³, about 10 ng/cm³ to about 900 ng/cm³, about 10 ng/cm³ to about 800 ng/cm³, about 10 ng/cm³ to about 700 ng/cm³, about 10 ng/cm³ to about 600 ng/cm³, about 10 ng/cm³ to about 500 ng/cm³, about 10 ng/cm³ to about 400 ng/cm³, about 10 ng/cm³ to about 300 ng/cm³, about 10 ng/cm³ to about 200 ng/cm³, or about 10 ng/cm³ to about 100 ng/cm³.

In some embodiments, the plant sensory compound is a secondary metabolite. In some embodiments, the plant sensory compound is tannin. In other embodiments, the plant sensory compound is selected from alkaloids, terpenoids, saponins, phenolic compounds, flavonoids, tannins, or a combination thereof.

It is believed that secondary metabolites in the plant extracts are responsible for the sensing capability because untargeted metabolomic analysis performed on plant extracts shows that secondary metabolites are the major compound in the plant extract. When used to form the sensing material, a partition coefficient of the sensing material is directly proportional to the VOC interaction with the sensing material thus increasing the change in the electrical properties (resistance and capacitance) of the sensor. It is believed that this occurs due to the secondary metabolite either electrostatically interacting with or complexing with the VOC.

For example, the plant sensory compound can be a tannin. Tannins (or tannoids) are a class of astringent, polyphenolic biomolecules that bind to and precipitate proteins and various other organic compounds including amino acids and alkaloids. Tannins can have molecular weights ranging from 500 to over 3,000 (gallic acid esters) and up to 20,000 Daltons (proanthocyanidins).

In some embodiments, the plant extract comprises tannin at a concentration of about 0.1 ng/cm³ to about 1000 μg/cm³. In other embodiments, the concentration is about 0.1 ng/cm³ to about 900 μg/cm³, about 0.1 ng/cm³ to about 800 μg/cm³, about 0.1 ng/cm³ to about 700 μg/cm³, about 0.1 ng/cm³ to about 600 μg/cm³, about 0.1 ng/cm³ to about 500 μg/cm³, about 0.1 ng/cm³ to about 400 μg/cm³, about 0.1 ng/cm³ to about 300 μg/cm³, about 0.1 ng/cm³ to about 200 μg/cm³, about 0.1 ng/cm³ to about 100 μg/cm³, about 0.1 ng/cm³ to about 90 μg/cm³, about 0.1 ng/cm³ to about 80 μg/cm³, about 0.1 ng/cm³ to about 70 μg/cm³, about 0.1 ng/cm³ to about 60 μg/cm³, about 0.1 ng/cm³ to about 50 μg/cm³, about 0.1 ng/cm³ to about 40 μg/cm³, about 0.1 ng/cm³ to about 30 g/cm³, about 0.1 ng/cm³ to about 20 μg/cm³, about 0.1 ng/cm³ to about 10 μg/cm³, or about 0.1 ng/cm³ to about 1 μg/cm³. In other embodiments, the concentration is about 1 ng/cm³ to about 1000 ng/cm³, about 1 ng/cm³ to about 900 ng/cm³, about 1 ng/cm³ to about 800 ng/cm³, about 1 ng/cm³ to about 700 ng/cm³, about 1 ng/cm³ to about 600 ng/cm³, about 1 ng/cm³ to about 500 ng/cm³, about 1 ng/cm³ to about 400 ng/cm³, about 1 ng/cm³ to about 300 ng/cm³, about 1 ng/cm³ to about 200 ng/cm³, or about 1 ng/cm³ to about 100 ng/cm³. In other embodiments, the concentration is about 10 ng/cm³ to about 1000 ng/cm³, about 10 ng/cm³ to about 900 ng/cm³, about 10 ng/cm³ to about 800 ng/cm³, about 10 ng/cm³ to about 700 ng/cm³, about 10 ng/cm³ to about 600 ng/cm³, about 10 ng/cm³ to about 500 ng/cm³, about 10 ng/cm³ to about 400 ng/cm³, about 10 ng/cm³ to about 300 ng/cm³, about 10 ng/cm³ to about 200 ng/cm³, or about 10 ng/cm³ to about 100 ng/cm³.

For example, the plant sensory compound can be an alkaloid. Alkaloids are a class of basic, naturally occurring organic compounds that contain at least one nitrogen atom. This group also includes some related compounds with neutral and even weakly acidic properties. In addition to carbon, hydrogen and nitrogen, alkaloids may also contain oxygen, sulfur and, more rarely, other elements such as chlorine, bromine, and phosphorus.

The plant extract is electrically conductive. This is believed to be due to the presence of ionisable or charged molecules within the plant extract, which can have molecular interactions (such as covalent bond, hydrogen bond) with the VOCs. In doing so, the electrical conductance or resistance of the plant extract is altered or varied. The change in the conductivity or resistivity may be used to characterise the VOC. Accordingly, by incorporating the plant extract into the sensing material of a chemiresistor, the chemiresistor may be made electrically conductive and may be used be used to characterise the VOC.

In some embodiments, the plant extract is characterised by a dielectric constant of about 1 to about 200. The dielectric constant (or relative permittivity) is the electric permeability of a material expressed as a ratio with the electric permeability of a vacuum. A dielectric is an insulating material, and the dielectric constant of an insulator measures the ability of the insulator to store electric energy in an electrical field. Permittivity is a material's property that affects the Coulomb force between two point charges in the material. Relative permittivity is the factor by which the electric field between the charges is decreased relative to vacuum. Likewise, relative permittivity is the ratio of the capacitance of a capacitor using that material as a dielectric, compared with a similar capacitor that has vacuum as its dielectric. In other embodiments, the dielectric constant is about 1 to about 180, about 1 to about 160, about 1 to about 140, about 1 to about 120, about 1 to about 100, about 1 to about 90, about 1 to about 80, about 1 to about 70, about 1 to about 60, about 1 to about 50, about 1 to about 40, or about 1 to about 30.

In some embodiments, the plant extract is characterised by a resistivity of about 100 Ω·m to about 8000 Ω·m. Electrical resistivity is a property of a material that measures how strongly it resists electric current. A low resistivity indicates a material that readily allows electric current. Electrical conductivity is the reciprocal of electrical resistivity. It represents a material's ability to conduct electric current. In other embodiments, the resistivity is about 100 Ω·m to about 7000 Ω·m, about 100 Ω·m to about 6000 Ω·m, about 100 Ω·m to about 5000 Ω·m, about 100 Ω·m to about 4000 Ω·m, about 100 Ω·m to about 3000 Ω·m, about 100 Ω·m to about 2000 Ω·m, about 100 Ω·m to about 1000 Ω·m, about 100 Ω·m to about 900 Ω·m, about 100 Ω·m to about 800 Ω·m, about 100 Ω·m to about 700 Ω·m, about 100 Ω·m to about 600 Ω·m, about 100 Ω·m to about 500 Ω·m, about 100 Ω·m to about 400 Ω·m, about 100 Ω·m to about 300 Ω·m, or about 100 Ω·m to about 200 Ω·m.

In some embodiments, the plant extract is characterised by a volatile organic compound partition coefficient. This parameter can be used to describe the solubility of the VOC in the sensing material. The partition coefficient is defined as a ratio, and has no unit. The partition coefficient (VOC in sensing material/VOC in gas) can be of about 0.1 to about 100. In other embodiments, the partition coefficient is about 1 to about 100, about 5 to about 100, about 10 to about 100, about 20 to about 100, about 30 to about 100, about 40 to about 100, about 50 to about 100, about 60 to about 100, about 70 to about 100, about 80 to about 100, or about 90 to about 100.

The plant extract may be a mixture of proteins, carbohydrates and other plant sensory compounds (biomolecules). It was found that impurities such as proteins, carbohydrates and/or macromolecules in the plant extract can decrease the responsiveness of the sensing material to VOC. This was further confirmed through the sensing abilities of pure proteins like Bovine serum albumin (BSA). It was found that these pure proteins are incapable of sensing VOCs. By removing these impurities, the sensing capability of the VOC sensor array can be improved. It also improves the response time and recovery time of the sensors. In this regard, the plant extract may be a purified plant extract.

In some embodiments, the plant extract has a protein concentration of less than about 30 mg/g. In other embodiments, the concentration is less than about 50 mg/g, about 45 mg/g, about 40 mg/g, about 35 mg/g, about 28 mg/g, about 26 mg/g, about 24 mg/g, about 22 mg/g, about 20 mg/g, about 18 mg/g, about 16 mg/g, about 14 mg/g, about 12 mg/g, about 10 mg/g, about 5 mg/g, or about 1 mg/g.

In some embodiments, the plant extract has a carbohydrate concentration of less than about 700 mg/g. In other embodiments, the concentration is less than about 1000 mg/g, about 900 mg/g, about 800 mg/g, about 650 mg/g, about 600 mg/g, about 550 mg/g, about 500 mg/g, about 450 mg/g, about 400 mg/g, about 350 mg/g, about 300 mg/g, about 250 mg/g, about 200 mg/g, about 150 mg/g, about 100 mg/g, about 50 mg/g, about 40 mg/g, about 30 mg/g, about 20 mg/g, about 10 mg/g, about 5 mg/g, or about 1 mg/g.

The sensing material can comprise other components to alter its final electrical resistance and/or capacitance. In some embodiments, the sensing material further comprises a polymer matrix. The polymer matrix provides support to the plant sensory compounds such that they are more spaced apart for receiving the VOC. The polymer matrix can be epoxy, polyurethanes, phenolic and amino resins, bismaleimides (BMI, polyimides), or polyamides.

In some embodiments, the sensing material is characterised by a thickness of about 0.1 μm to about 1000 μm. The thickness can be an initial thickness, i.e. before the permeation of the VOC into the sensing material. When the sensing material interacts with the VOC, the permeation of the VOC into the sensing material can cause the thickness of the sensing material to increase, thereby resulting in change in the electrical properties. In other embodiments, the thickness is about 0.1 pm to about 900 μm, about 0.1 μm to about 800 μm, about 0.1 μm to about 700 μm, about 0.1 μm to about 600 μm, about 0.1 μm to about 500 μm, about 0.1 μm to about 400 μm, about 0.1 μm to about 300 μm, about 0.1 μm to about 200 μm, about 0.1 μm to about 100 μm, about 0.1 μm to about 50 μm, or about 0.1 μm to about 10 μm.

In some embodiments, each of the sensing material is independently characterised by a dielectric constant of about 1 to about 200. In other embodiments, the dielectric constant is about 1 to about 180, about 1 to about 160, about 1 to about 140, about 1 to about 120, about 1 to about 100, about 1 to about 90, about 1 to about 80, about 1 to about 70, about 1 to about 60, about 1 to about 50, about 1 to about 40, or about 1 to about 30.

In some embodiments, each of the sensing material is independently characterised by a resistivity of about 100 Ω·m to about 8000 Ω·m. In other embodiments, the resistivity is about 100 Ω·m to about 7000 2·m, about 100 Ω·m to about 6000 Ω·m, about 100 Ω·m to about 5000 Ω·m, about 100 Ω·m to about 4000 Ω·m, about 100 Ω·m to about 3000 Ω·m, about 100 Ω·m to about 2000 Ω·m, about 100 Ω·m to about 1000 Ω·m, about 100 Ω·m to about 900 Ω·m, about 100 Ω·m to about 800 Ω·m, about 100 Ω·m to about 700 Ω·m, about 100 Ω·m to about 600 Ω·m, about 100 Ω·m to about 500 Ω·m, about 100 Ω·m to about 400 Ω·m, about 100 Ω·m to about 300 Ω·m, or about 100 Ω·m to about 200 Ω·m.

In some embodiments, the resistance is about 100 Ohm to about 10 M Ohm. The pattern formed by the change in the resistance across all the sensors in the sensor array may be used for the sample detection and the magnitude of the change in resistance may be used to quantify the sample concentration.

In some embodiments, each of the sensing material is independently characterised by a volatile organic compound partition coefficient. The partition coefficient (VOC in sensing material/VOC in gas) can be of about 0.1 to about 100. In other embodiments, the partition coefficient is about 1 to about 100, about 5 to about 100, about 10 to about 100, about 20 to about 100, about 30 to about 100, about 40 to about 100, about 50 to about 100, about 60 to about 100, about 70 to about 100, about 80 to about 100, or about 90 to about 100.

In some embodiments, the VOC sensor array further comprises a conductive material sandwiched between and in electrical communication with the chemisresistor array and the electronic means. The conductive material further improves the electrical contact between the sensing material and the electrodes.

In some embodiments, the sensing material is coated on the conductive material. The sensing material may be a layer or a film on the conductive material.

In some embodiments, the conductive material is wire glue, carbon grease, carbon nanomaterial, or a combination thereof. The carbon nanomaterial can be graphene, carbon nanotube, or a combination thereof. The conductive material may be applied as required to improve the conductance and/or electrical contact between the sensing material and the electrodes.

With at least 3 chemiresistor, it was found that the VOC sensor array may be highly specific. For example, yogurt of different flavours may be distinguished.

The number of chemiresistors can be increased by independently incorporating various other plant and/or flower extracts to increase the dimensionality, thus increasing the resolution of these sensor arrays. For example, the sensor array can comprise 4 chemiresistors, each having a sensing material comprises a different plant extract. Accordingly, the response to a VOC can be quantified via the chemiresistive responses from the 4 different chemiresistors. In some embodiments, the chemiresistor array comprises at least 2 chemiresistors, or at least 3 chemiresistors. In other embodiments, the chemiresistor array comprises at least 4 chemiresistors, at least 5 chemiresistors or at least 6 chemiresistors. In other embodiments, the chemiresistor array comprises more than 1 chemiresistor.

In some embodiments, the space is an enclosed space. The enclosed space ensures that the VOC does not escape the proximity of the sensors and that an accurate reading can be obtained.

To further improve the responsiveness of the VOC sensor array, the space above the chemiresistor array can be configured to allow circulation of the VOC within the space. For example, the space can be provided with circulation means, such as a fan to ensure that the VOC is homogenously dispersed within the space. The space can be limited to a certain volume, for example about 1 cm³ to about 10 cm³. This reduces the amount of VOC required for interaction with the sensing material. In other embodiments, the volume is about 1 cm³ to about 9 cm³, about 1 cm³ to about 8 cm³, about 1 cm³ to about 7 cm³, about 1 cm³ to about 6 cm³, about 1 cm³ to about 5 cm³, about 1 cm³ to about 4 cm³, about 1 cm³ to about 3 cm³, or about 1 cm³ to about 2 cm³.

In some embodiments, the electronic means comprises a voltage divider.

In some embodiments, the electronic means comprises a microcontroller. A microcontroller (MCU for microcontroller unit) is a small computer on a single metal-oxide-semiconductor (MOS) integrated circuit (IC) chip. A microcontroller contains one or more CPUs (processor cores) along with memory and programmable input/output peripherals. Program memory in the form of ferroelectric RAM, NOR flash or OTP ROM is also often included on chip, as well as a small amount of RAM. In some embodiments, the VOC sensor array comprises of an interdigitated electrode arrays. In some embodiments, the VOC sensor array comprises of a rigid substrate. The rigid substrate may be a printed circuit board (PCB).

In some embodiments, the change in electrical property is a change in resistance. It is believed that this is due to the change in thickness of the sensing material as the VOC percolates into the sensing material with respect to its partition coefficient in the sensing plant extract. With an increase in VOC penetrating the sensing material, the resistance provided by the sensing material increases. The change in resistance before and after exposure to VOC can be used to quantify and/or qualify the VOC.

In some embodiments, the change in electrical property is characterised by a change in a thickness of the sensing material.

The VOC sensor array can also be used in the capacitance mode. In this regard, the VOC sensor array can be versatile in the mode of measurement, and can provide two types of data from one sensing material provide. This gives it additional robustness and reliability in the gas sensing applications.

In some embodiments, the VOC sensor array further comprises a dielectric layer. The dielectric layer can be sandwiched between the sensing material and the electrodes or adjacent to the electrodes. The dielectric layer allows for capacitance sensing.

The capacitance of the sensors can be measured using an electrical circuit to deduce the unknown capacitance by measuring the time required to charge the capacitor. By measuring the change in the capacitance values of the sensor array after the introduction of the analyte, these value from each sensors in the array is used to create a plots like radar plot which will be distinct for the particular analyte.

In some embodiments, the change in electrical property is a change in capacitance.

In some embodiments, the change in electrical property is obtainable after about 1 min to about 120 min. In other embodiments, the time is about 1 min to about 100 min, about 1 min to about 90 min, about 1 min to about 80 min, about 1 min to about 70 min, about 1 min to about 60 min, about 1 min to about 50 min, about 1 min to about 40 min, about 1 min to about 30 min, about 1 min to about 20 min, about 1 min to about 10 min, about 1 min to about 5 min, about 40 min to about 120 min, about 50 min to about 120 min, about 60 min to about 120 min, about 70 min to about 120 min, about 80 min to about 120 min, or about 90 min to about 120 min.

In some embodiments, the VOC sensor array further comprises a sample holder for containing a sample, the sample holder in fluid communication with the space. The sample holder can be a container with air space.

In some embodiments, the sample holder comprises a heating means. The heating means can be positioned adjacent to the sample.

In some embodiments, the sample is passed through an atomiser. The atomiser micronizes the sample into fine droplets to increase the surface area. The fine droplets may then be passed to a heating means for quick conversion of liquid samples into VOCs.

In some embodiments, the VOC sensor array further comprises a substrate. In some embodiments, the VOC sensor array further comprises a flexible substrate. In some embodiments, the flexible substrate is a thermoplastic polymer sheet, for example, polyethylene terephthalate (PET) sheet. In some embodiments, the VOC sensor array further comprises a rigid substrate. In some embodiments, the rigid substrate is a printed circuit board (PCB).

In some embodiments, the VOC sensor array further comprises a controller. The controller may control a sequential flow of events for sensing and/or analysing the VOC. As will be understood, a controller will generally be embodied by electronic components, particularly electronic components programmed to facilitate control of a sequential flow of reagents and/or sample into VOC sensor array, and other necessary functions. For example, the controller may be configured to determine the amount of sample in the sample holder, aliquot an appropriate amount to the chemisresistor for sensing, activate the heating means and determining the electrical conductivity of the chemiresistor.

The VOC sensor may further comprise quantifying means in order to quantify the amount and type of VOC in the sample.

In some embodiments, VOC sensor array is reusable. In this regard, the interaction between the sensing material and VOC is reversible.

In use, the VOC sensor array with the sensing material in its initial state (no VOC partitioned within) is contacted with a sample. The sample releases VOC into the space and which partitions into the sensing material. Over time, as more VOC is released into the space, the electrical signal derived from the VOC sensor array increases. When an equilibrium state between the VOC in the space and the VOC in the sensing material is reached, the electrical signal derived from the VOC sensor array reaches a constant. The VOC sensing array can be reused by purging the space with an inert gas or air for a period of time. This allows the sensing material to return to its initial state and another sample can be contacted.

When a VOC is introduced, the VOC sensor array may reach equilibrium with the VOC in about 1 min to about 30 min. In other embodiments, equilibrium is reached about 5 min to about 30 min, about 10 min to about 30 min, or about 15 min to about 30 min.

As the VOC sensor array comprises one or more than one sensor with more than one type of sensing material, a response pattern is obtainable which is specific to the sample. For example, a spider map may be used to characterise the VOC.

In some embodiments, the VOC sensor array is characterised by a sensitivity of about 80% to about 100%. In other embodiments, the sensitivity is about 85% to about 100%, about 90% to about 100%, or about 95% to about 100%.

The present disclosure also provides a method of fabricating a volatile organic compound (VOC) sensor array as disclosed herein, comprising:
a) casting the plant extract as a layer on the electronic means; and
b) drying the plant extract in order to form the sensing material in electrical communication with the electronic means.

The electronic means may be electrodes.

In some embodiments, the method of fabricating a volatile organic compound (VOC) sensor array as disclosed herein comprises:
a) casting the plant extract as a layer on the conductive material; and
b) drying the plant extract in order to form the sensing material in electrical communication with the conductive material.

The plant extract can be provided as a solution. The plant extract can be drop casted onto the conductive material, or spin coated onto the conductive material or electrodes. The drying step removes the solvent in the solution of plant extract.

The plant extract may be mixed with a conducting polymer, composite or metal oxides as mentioned above before casting on the conductive material. The plant extract may be homogenously mixed with the conducting polymer, composite or metal oxides before casting. In this way, the plant extract is dispersed within the sensing material and may provide a consistent output.

In some embodiments, the extraction step comprises:
a) crushing a plant sample and incubating the plant sample in a polar solvent;
b) separating the plant sample of step a) into a supernatant and a residue; and
c) extracting the supernatant in order to form the plant extract.

The crushing step breaks down the plant sample into smaller pieces such that the solvent can more easily penetrate the plant and extract the biomolecules.

In some embodiments, the polar solvent is ethanol. Ethanol was found to be particularly advantageous as it is miscible with water, and can extract polar secondary metabolites. It is also a self-preservative at a concentration above 20%. It is nontoxic at low concentration, and as small amount of heat is required for concentrating the extract.

Other examples of solvents that can be used are water, methanol, n-butanol, acetone, isopropanol, nitromethane, acetic acid, and formic acid. Ionic liquid (green solvent) can also be used. It has extreme miscibility with water and other solvent and is very suitable in the extraction of polar compounds. It has excellent solvent that attracts and transmit microwave, and hence it is suitable for microwave-assisted extraction. It is non-flammable and is useful for liquid-liquid extraction and highly polar.

The plant extract can be used as is, or it can be further purified.

In some embodiments, the extraction step further comprises:
d) incubating the plant extract in an aprotic solvent;
e) separating the plant extract of step d) into a supernatant and a residue; and
f) extracting the supernatant in order to form a purified plant extract.

An aprotic solvent is a solvent that contains a non-labile H⁺ and is thus incapable of acting as a proton donor. The molecules cannot form H-bonds with themselves, but they may accept H-bonds from other molecules. In some embodiments, the aprotic solvent is acetonitrile.

Advantageously, no heating is involved in the extraction method, and thus the biomolecules are not denatured.

In some embodiments, the method further comprises providing a space adjacent to the sensing material in each chemiresistor.

In some embodiments, the method further comprises electrically connecting the conductive material to electronic means.

The response can be further improved by normalising the plant extract against a standard and/or a control. In some embodiments, the control is a total protein concentration. In other embodiments, the control is a weight ratio. For example, the plant extract can be normalised against the weight of the plant sample.

In some embodiments, the method further comprises a step of normalising the plant extract or purified plant extract against a standard and/or a control.

In some embodiments, the control is a total protein concentration. In some embodiments, the control is a weight ratio relative to the weight of the plant sample.

The present disclosure also provides a method of extracting plant based extracts.

The method of extraction eliminates other biochemical compounds that does not have the gas sensing capabilities. The extraction method can provide robust, low-noise and reproducible results. The extraction method also decreases the response time as well as the recovery time.

### EXAMPLES

### General Plant Extraction Method

1. Grind the plant material using a polar solvent and collect the extracts. Any solvent can be used. In particular, ethanol was found to be advantageous.
2. Precipitate or remove the proteins and macromolecules from the extract and collect the non-precipitated liquid fraction. In particular, acetonitrile was found to be advantageous but any solvent or solution that can remove macromolecules like proteins, nucleic acids etc., can be used.
3. Dry the collected liquid fraction.
4. Re-dissolve the dried fraction with polar solvents and remove the un-dissolved materials which leave the dissolved solution which is the sensing material.

### Extraction Method 1

While any plant species and any plant part can be used to fabricate chemiresistor, two plant species were selected to exemplify the working mechanism of the invention: Caesalpinia pulcherrima (C. pulcherrima) and Ixora coccinea (I. coccinea). Sepals and petals from the Caesalpinia pulcherrima were separated and used to make two chemiresistors one with sepals and other with petals. With Ixora coccinea, only the petals were used to make chemiresistors. The flower materials of these two plants were freshly collected and ground with a pestle as soon as possible. Ethanol was added as the extraction solvent, followed by further grinding of the flower material with ethanol. In general, 1 g of plant material is dispersed in 1 mL of solvent. The extract was separated from the plant debris by centrifugation at 10,000 rpm for 15 mins. The supernatants collected were used for forming the sensing material of the chemiresistors. The supernatant (about 100 μL) was drop casted on to the conductive layer.

The plant extract produced in this method has a protein concentration of more than about 20 μg/cm³. The plant extract produced in this method has a carbohydrate concentration of more than about 20 μg/cm³.

### Extraction Method 2

Flower's petals were separated and crushed using micro pestle. Absolute ethanol was added as the extraction solvent and crushed using micro pestle. The crushed petals in ethanol was incubated. After incubation the samples were vortexed and centrifuged. The supernatant was saved and the pellet was discarded. In order to normalize the extracts collected from different plant materials, protein concentration of the supernatant were estimated using bradford's protein assay. The supernatant volume equivalent to a specific quantity of protein was aliquoted and Acetonitrile was added at 1:2 or 1:3 protein:ACN ratio in order to precipitate the protein impurity. This extract was incubated. After incubation, the samples were vortexed and centrifuged. The supernatant was saved and the pellet was discarded. Dry the supernatant in vacuum. Dissolve the dried pellet in absolute ethanol using sonicator. After incubation the samples were vortexed and centrifuged. The supernatant was saved and the pellet was discarded. The supernatant is then normalised to correspond to a protein quantity in order to reduce batch wise variability. The supernatant (about 20 μL-50 μL) was drop casted on to the conductive layer.

### Fabrication of the Sensor

Though any conducting material can be used, for the demonstration purposes commercially available wire glue is used as the base conductive layer. Wire Glue is an electrically conductive, water-based adhesive. PET sheets are cut into required dimensions and terminals were created using copper strips and connecting pins. The plant based sensing material (about 10 μL) is drop casted on the base layer (either conductive layer (wire glue) or directly on the interdigitated electrodes). Carbon grease can also be used as the conductive layer.

For the control sensor, ethanol (extraction solvent) alone was drop casted on the base layer. Four sensors were mounted on the plastic lid a sensor array. Examples of gas sensors arrays are shown in FIG. 3.

### Sensing Milk to Yogurt Conversion

To test the chemiresistive functionality of the chemiresistors, these sensors were tested to determine if they could detect the milk spoilage. 5 mL of cow's milk is taken from the 1 L milk pack bought from the local supermarket. A 100 μL of yogurt is added to the 10 mL milk followed by closing it with a lid on which the sensor array was mounted. The resistance of the sensors at time zero was deduced and noted. Resistance readings were recorded for every 1 minute till the total duration of 12 hours using the voltage divider circuit connected to the micro controller (Arduino Mega 2650). This entire experiment is repeated thrice, and the change in resistance is calculated from the obtained resistance readings (Eq. 1).

$$Δ R = {R}_{t} - {R}_{0} \qquad Eq . 1$$

### Chemiresistive Sensing of Different Flavors of Smoothies

The ability of the florisensor array to sense different flavors of smoothies were accessed. 5 mL of smoothie is taken in a glass beaker, and the beaker is closed with the lid on which the gas sensor array is mounted. The resistance readings of the sensor array were recorded every 1 minute using the voltage divider circuit connected to the micro controller (FIG. 2). This entire experiment was repeated thrice for each smoothie flavor, and the change in resistance is calculated from the obtained resistance readings (Eq. 1).

### Discriminating Different Flavors of Smoothies

The differential resistance (ΔR) values from the recorded resistance values were calculated followed by deducing the mean differential resistance and standard deviation.

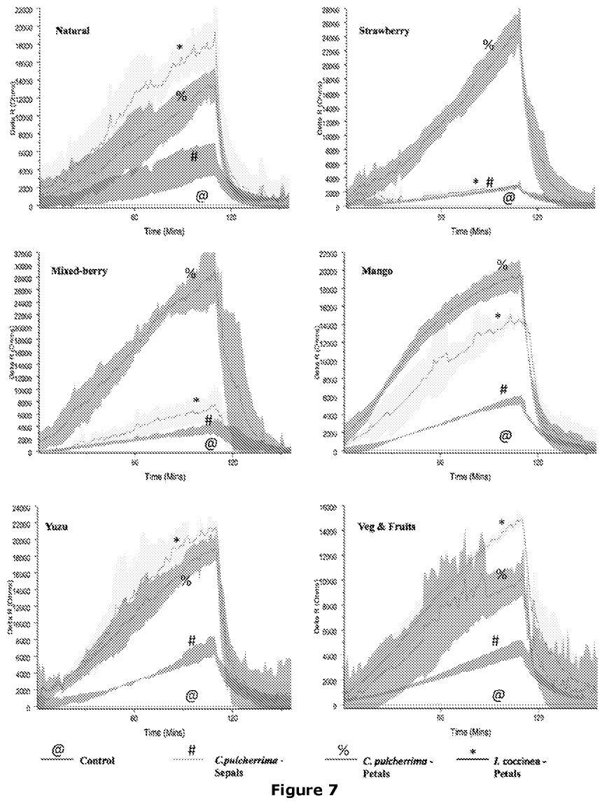

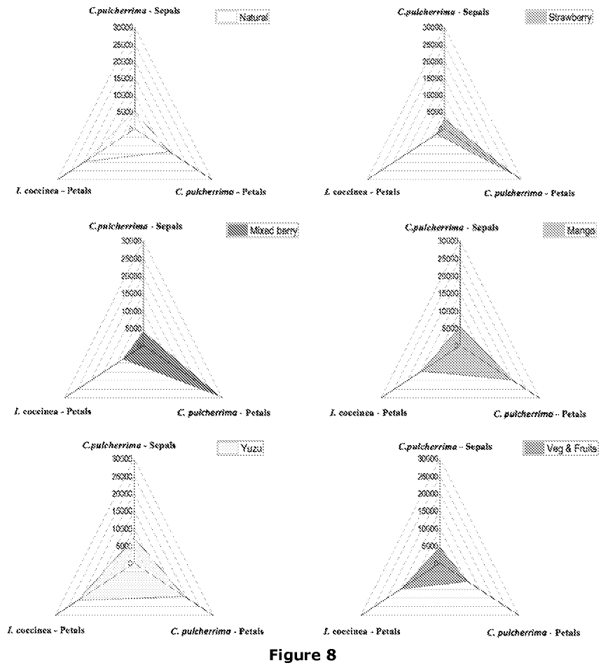

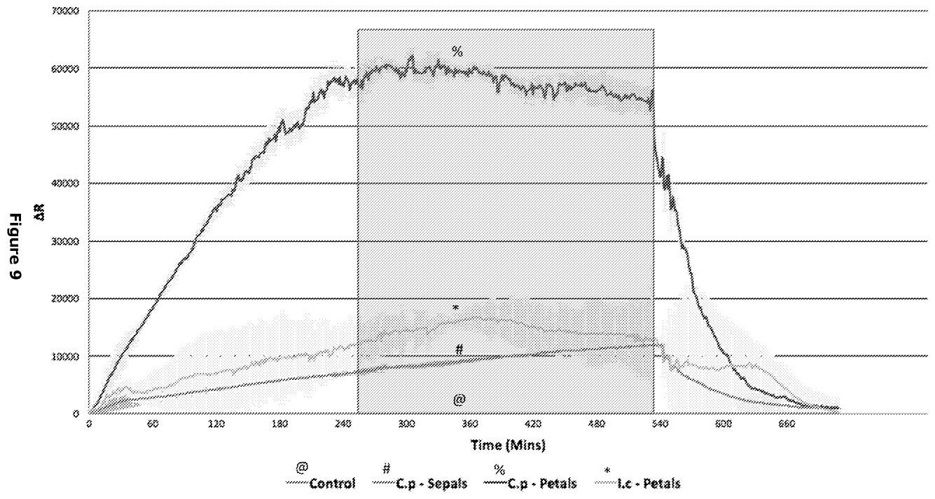

FIG. 7 shows the ΔR responses of all the four sensors to the headspace volatiles of different flavours of smoothies. The responses of the chemiresistors vary with the various flavours of smoothies and the control sensors did not show any significant variation, thus proving the chemiresistive properties of these chemiresistors. It is also observed that each flavour provoked distinct response pattern in the gas sensor array, which suggests that, it is possible to discriminate different flavours of the smoothie based on the response pattern of the gas sensors array. Radar chart was plotted using the AR values at which the sensor responses get saturated to visualize these distinct patterns, (FIG. 8). As shown, each flavour of the smoothie shows a distinct pattern in the radar chart. Thus the responses from at least three chemiresistors were sufficient to discriminate all the 6 flavours of the smoothie tested.

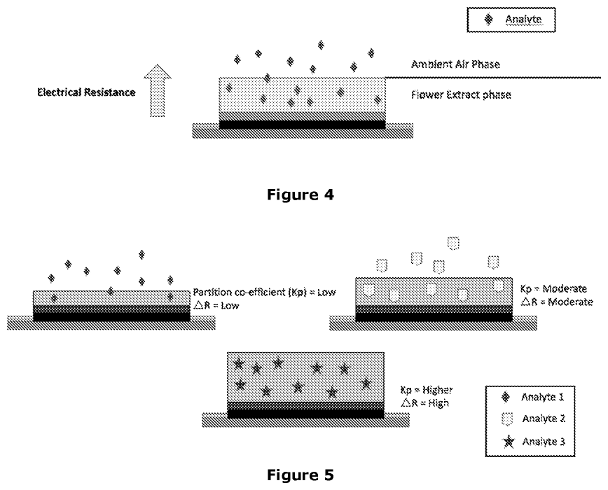

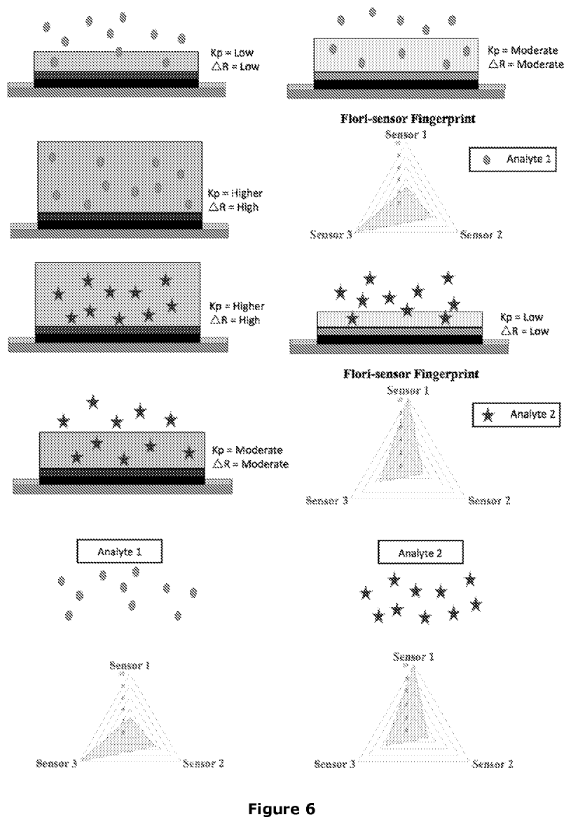

The variation in the resistances when exposed to the smoothies was attributed to the percolation of the volatiles into the sensing layer causing swelling and resulting in the resistance change as shown in FIG. 4. The specificity of these sensors was attributed to the partition coefficient of the volatiles in the sensing layer. If the exposed volatiles have high partition coefficient to the sensing layer, more volatiles percolate into the sensing layer causing more swelling resulting more change in resistance as shown in FIG. 5. Whereas, if the partition coefficient is low to the sensing layer, only few volatiles percolate causing less swelling leading to the minor change the resistance. Here, the partition coefficient depends on the plant extract which is used as the sensing layer. Hence, different plant extract responds differently to different volatiles thus creating distinct response patterns as shown in FIG. 6. The control sensor does not respond to the volatiles. Hence the partition coefficient of these volatiles in this sensor should be near zero.

### Milk to Yogurt Conversion

FIG. 9 shows the responses of the VOC sensor array in response to the conversion of milk to yogurt. From this plot, it was evident from the sensors drop-casted with C. pulcherrima petals and I. coccinea petals that milk to yogurt conversion completes in about 4 hours. The C. pulcherrima sepals based chemiresistor does not saturate and tend to have a linear curve. This data shows that this sensor does not have specificity towards the volatiles produced due to yogurt formation. Thus, this sensor allows percolation of all the volatiles in the headspace resulting in the linear curve as the concentration of the headspace volatiles would increase with the yogurt formation. However, this sensor indicates the continuous changes undergone by the analyte (Milk) due to the yogurt inoculation. It is important to note that these sensors tend to recover spontaneously when the volatiles were removed. This is in contrast to nanoparticle based sensors which need other external stimuli like UV exposure, thermal energy for sensor recovery. This feature makes these sensors more energy efficient over those expensive and energy inefficient chemiresistive sensors.

### Specificity and Sensitivity

The specificity of the chemiresistors in identifying different analyte has been demonstrated, as VOC sensor array can detect and distinguish between different flavors of yogurt smoothies with the sensor fingerprints. The specificity of the VOC sensor array could be further improved by adding more chemiresistors in the array, thus increasing the dimensionality of the sensor fingerprints. The reproducibility of the VOC sensor array (n=3) along with standard deviation values at each data point has shown in FIG. 7 and FIG. 9.

### Conclusion

The capability of plant extracts to act as the chemiresistive gas sensors is shown. These chemiresistive sensors are made from renewable source. These sensor responses were demonstrated to be reproducible as the standard deviation values were less enough to discriminate between different samples. The sensor fabrication is straightforward, easy, does not use hazardous or environment detrimental chemicals and low cost. This study shows that these chemiresistors are reusable and applicable to various real-time situations where VOC or gas sensing is indispensable.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e., necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

1. A volatile organic compound (VOC) sensor, comprising:
a) a chemiresistor comprising a sensing material;
b) a space adjacent to the sensing material in the chemiresistor, the space for providing the VOC to the sensing material; and
c) electronic means in electrical communication with the chemiresistors in order to detect a change in an electrical property of the sensing material upon exposure to the VOC;
wherein the sensing material independently comprises a plant extract;
wherein the plant extract comprises a plant sensory compound at a concentration of about 1 ng/cm³ to about 100 μg/cm³; and
wherein each of the plant extract is electrically resistive and/or electrically capacitive, the electrical resistance and/or electrically capacitive is configured to change in response to the VOC in the space.

2. A volatile organic compound (VOC) sensor array, comprising:
a) a chemiresistor array, each chemiresistor comprising a sensing material;
b) a space adjacent to the sensing material in each chemiresistor, the space for providing the VOC to the sensing material; and
c) electronic means in electrical communication with the chemiresistors in order to detect a change in an electrical property of the sensing material upon exposure to the VOC;
wherein each of the sensing material independently comprises a plant extract;
wherein the plant extract comprises a plant sensory compound at a concentration of about 1 ng/cm³ to about 100 μg/cm³; and
wherein each of the plant extract is electrically resistive and/or electrically capacitive, the electrical resistance and/or electrically capacitive is configured to change in response to the VOC in the space.

3. The VOC sensor array according to claim 2, wherein the plant sensory compound has a concentration of about 1 ng/cm³ to about 1 μg/cm³.

4. The VOC sensor array according to claim 2, wherein each of the plant extract is independently characterised by a dielectric constant of about 1 to about 200.

5. The VOC sensor array according to claim 2, wherein the plant extract comprises a protein at a concentration of less than about 30 mg/g and/or a carbohydrate concentration of less than about 700 mg/g; and/or wherein the plant extract comprises tannins at a concentration of about 0.1 g/cm³ to about 1000 μg/cm³/cm³.

6. The VOC sensor array according to claim 2, wherein the plant extract is derived from a flowering plant selected from Caesalpinia pulcherrima, Ixora coccinea, or a combination thereof.

7. The VOC sensor array according to claim 2, wherein the plant extract is derived from plant part selected from sepal, petal, leaf, root, or a combination thereof.

8. (canceled)

9. The VOC sensor array according to claim 2, wherein the sensing material is characterised by a thickness of about 0.1 μm to about 100 μm.

10. The VOC sensor array according to claim 2, wherein the VOC sensor array further comprises a conductive material sandwiched between and in electrical communication with the chemisresistor array and the electronic means;
wherein the conductive material is wire glue, carbon grease, carbon material (such as graphene, carbon nanotube), or combinations thereof.

11. (canceled)

12. The VOC sensor array according to claim 2, wherein the chemiresistor array comprises at least 2 chemiresistors.

13. (canceled)

14. (canceled)

15. (canceled)

16. The VOC sensor array according to claim 2, wherein the change in electrical property is a change in resistance and/or capacitance.

17. The VOC sensor array according to claim 2, wherein the change in electrical property is obtainable after about 1 min to about 120 min.

18. (canceled)

19. (canceled)

20. The VOC sensor array according to claim 2, wherein the VOC sensor array further comprises a flexible substrate or a rigid substrate, wherein the flexible substrate is a polyethylene terephthalate (PET) sheet and the rigid substrate is printed circuit board (PCB).

21. The VOC sensor array according to claim 2, wherein the VOC sensor array is characterised by a sensitivity of about 80% to about 100%.

22. A method of fabricating a volatile organic compound (VOC) sensor or volatile organic compound (VOC) sensor array according to claim 1, comprising:
a) casting the plant extract as a layer on the electronic means; and
b) drying the plant extract in order to form the sensing material in electrical communication with the electronic means.

23. The method according to claim 22, wherein the extraction step comprises:
a) crushing a plant sample and incubating the plant sample in a polar solvent;
b) separating the plant sample of step a) into a supernatant and a residue; and
c) extracting the supernatant in order to form the plant extract.

24. The method according to claim 23, wherein the extraction step further comprises:
d) incubating the plant extract in an aprotic solvent;
e) separating the plant extract of step d) into a supernatant and a residue; and
f) extracting the supernatant in order to form a purified plant extract.

25. The method according to claim 22, wherein the method further comprises providing a space adjacent to the sensing material in each chemiresistor.

26. The method according to claim 22, wherein the method further comprises electrically connecting the conductive material to electronic means.

27. The method according to claim 22, wherein the method further comprises a step of normalising the plant extract or purified plant extract against a standard and/or a control;
wherein the control is a total protein concentration and/or a weight ratio relative to the weight of the plant sample.

28. (canceled)

29. (canceled)
